(19) United States
(12) Patent Application Publication
(10) Pub. No.: US 2023/0270912 A1
(43) Pub. Date: Aug. 31, 2023

(54) PERACETIC ACID-BASED FORMULATION ASSOCIATED WITH A GRINDING PROCESS, THE COMBINATION OF WHICH TRANSFORMS CULTURES AND STRAINS OF BIOHAZARDOUS INFECTIOUS WASTE GENERATED IN THE PRODUCTION OF VACCINES IN OVO INTO RAW MATERIAL FOR THE PREPARATION OF HIGH-PROTEIN COMPOSTS

(71) Applicant: CENTRO DE RETINA MÉDICA Y QUIRÚRGICA, S.C., Zapopan, Jalisco (MX)

(72) Inventors: Arturo SANTOS GARCÍA, Zapopan, Jalisco (MX); Gisela Hialita SÁNCHEZ SOSA, Zapopan, Jalisco (MX); Juan Pablo AGUILAR ALEMÁN, Zapopan, Jalisco (MX)

(21) Appl. No.: 18/015,794

(22) PCT Filed: Jun. 3, 2021
(86) PCT No.: PCT/MX2021/000020

§ 371 Date: Jan. 12, 2023

(30) Foreign Application Priority Data
Jul. 13, 2020 (MX) MX/A/2020/005776

(51) Int. Cl. A61L 11/00; A01N 37/16; A61L 2/18; B09B 3/35; B09B 3/70; C05F 7/00; C05F 1/00

(52) U.S. Cl. CPC A61L 11/00; A01N 37/16; A61L 2/18; B09B 3/35; B09B 3/70; C05F 1/00; C05F 7/00; B09B 2101/65

(57) ABSTRACT

Disclosed is a formulation which, in association with a grinding system, transforms biohazardous wastes unto raw material for the preparation of composts, enabling biohazardous infectious waste to be inactivated for use and industrial biohazardous infectious waste to be inactivated for reuse as a protein source for producing protein products. The formulation can be used to eliminate microorganisms in waste resulting from the production of drugs and vaccines in ovo, with applications in biohazardous infectious waste and animal waste with a high protein content and decomposition potential for the preparation of composts.

### CROSS REFERENCE TO RELATED APPLICATIONS

This application is a National Stage Application of PCT International Application No. PCT/MX2021/000020 under 35 U.S.C. § 371(a), filed Jun. 3, 2021, which claims the benefit of, and priority to, Mexican Application No. MX/a/2020/005776, filed Jul. 13, 2020, the entire contents of each of which are hereby incorporated herein by reference.

### FIELD OF THE INVENTION

The present invention is related to the field of biotechnology and health, specifically to a peracetic acid-based formulation associated with a grinding process, the combination of which produces a highly effective bactericidal, fungicidal, and virucidal action which transforms cultures and strains of biohazardous agents (biohazardous waste) generated in the production of vaccines in ovo into special wastes and enables the same to be reused as raw material for the preparation of high-protein composts.

### BACKGROUND OF THE INVENTION

Biohazardous wastes are those materials generated during medical care services, at funeral homes, biological companies, inter alia, which contain biohazardous agents and can cause harmful effects to health and the environment.

The treatment of biohazardous waste is currently carried out using different methods, with the most commonly used being incineration, sterilization by autoclaving, application of microwaves, and finally sterilization or chemical disinfection. With regards to chemical sterilization, various disinfectants can be used, such as chlorine dioxide, sodium hypochlorite, ethylene oxide, formaldehyde gas, glutaraldehyde, or peracetic acid. Peracetic acid has shown great biocidal capacity, and the compounds are completely innocuous when disposed of in the environment. However, no peracetic acid formulation has been reported which, in combination with a grinding process, enables this type of waste to be reused as raw material for the preparation of composts with a high protein content.

Peracetic acid, also called peroxyacetic acid or simply PAA, is known for its high oxidative capacity. This compound has a condensed formula of CH₃COOOH and a molecular mass of 76.05 g/mol. This aggregate has a pungent odor, is clear, and is normally diluted with water when used as a sterilizing agent. The most common aqueous formulations contain peracetic acid in a concentration of between 5, 15, and 35 percent with respect to the total solution, and it is formulated in the presence of acetic acid and hydrogen peroxide; these two compounds are in chemical equilibrium and form PAA. Concentrations below 5% have been observed in broad antimicrobial and biocidal activity, and studies exist which demonstrate the elimination of bacteria and fungi, primarily on surfaces. Nevertheless, the percentage characterization that is to be employed and the effect thereof on viruses is very limited.

Due to its oxidizing capacity and rapid action, peracetic acid in dilution with hydrogen peroxide has unique advantages over other disinfectants. The environmental impact of this mixture is non-existent, since it hydrolyzes readily and very quickly (between 20 to 25 minutes at a low concentration in water) into acetic acid and oxygen. At present, this component is used in the food industry for cleaning fruit, vegetables, and meat processing rooms. However, given its great germicidal capacity against virus spores, bacteria, and fungi, it is used for the sterilization of surgical instruments and virus culture areas in pharmaceuticals, the latter representing an advancement over discarding aldehydes as sterilants.

Finally, PAA offers the possibility of being used for the sterilization of biohazardous waste, and when it is combined with a destruction process (leaving them unrecognizable and increasing the biocidal efficacy of the PAA by exposing non-surface matter and producing a reaction mixture), biohazardous waste treated in this manner may come to be classified as special waste, thereby enabling this waste to be utilized as raw material for the preparation of compost. An alternative with no environmental impact, a circular process, and the elimination of industrial waste would thus be achieved.

### Previous Solutions

The reuse of waste of organic origin enables the waste to be transformed into composts. Various solutions have been described in which peracetic acid is used as a degrading agent for the purpose of enabling compost to be produced from organic systems. Likewise, wastes or residues of an industrial nature, which tend to originate from the petrochemical industry, can be reused due to the presence of a system that combines peracetic acid. However, no solution is known which allows for the inactivation of biohazardous waste and reuse thereof as raw material for the preparation of compost with a high protein content or other organic-based products that can be used in different industrial sectors.

### Prior Art

Based on the analysis of prior art documents, there are inventions that attempt to solve similar problems, as is the case with the invention described in document WO2009040447A1, which discloses a method for conditioning compost using a solution of a peroxyacetic mixture. The conditioning process consists of saturating the substrate with water and subsequent washing with a volume of an aqueous solution of peroxyacetic mixture of between 1 and 10 times the volume of the compost. The peroxyacetic mixture is an aqueous solution of hydrogen peroxide and peracetic acid in different proportions. The concentration of peroxyacetic mixture in the aqueous solution that we propose for use as compost conditioning is between 1-10%. The concentration of hydrogen peroxide (H₂O₂) in the peroxyacetic mixture is between 10-40%, and the concentration of peroxyacetic acid (C₂H₄O₃) is between 1-10%. The method for conditioning the compost with a peroxyacetic mixture for direct use as an agronomic and forestry substrate has the objective of obtaining high-quality compost, even with the possibility of being an alternative to Sphagnum peat, with the added incentive that the environmental and economic cost of this process is lower than the other alternatives. The invention disclosed herein differs from this in many respects; the formulation is different, and the application of this prior art is oriented toward compost, whereas the invention which is the subject of the present document is oriented toward biohazardous waste generated in the production of vaccines in ovo.

On the other hand, the invention JP2003334531A of May 17, 2002 describes a centralized treatment system which integrally comprises the treatment of non-industrial waste, medical waste, and industrial waste for use as a resource. The centralized treatment system includes a first step of providing a treatment center for every 200,000 to 300,000 people; a second step, which consists of separating and recovering three types of waste; a third step, which consists of burning non-industrial waste and utilizing the residual heat in a power generation device, crop plantations, or the like; a fourth step, which consists of separating waste plastics from industrial waste; a fifth step, which is to liquefy the remaining plastic waste using the produced oil and the like to generate power in a power generating apparatus and supply the waste heat to a crop plantation or the like; a sixth step, which is to pass a waste oil together with another waste oil through a waste oil mixing apparatus and treat the waste oil in a liquefaction apparatus in order to generate a produced oil; a seventh step, which consists of producing compost, food, reagents, and the like from organic industrial waste; an eighth step, which consists of treating batteries, fluorescent lamps, heavy metals, and ashes burned in an incinerator, waste in the liquefaction apparatus, and the like, in a smelting furnace; and a ninth step, which is to use pig urine, cattle urine, and the like as raw materials and to treat the raw materials in a fermentation tank and an ionization tank in order to produce a deodorant and ionized drinking water. That invention differs from that disclosed herein by virtue of its formulation and applications.

The invention WO1999047282A1 of Sep. 23, 1999 describes a method for treating infectious waste organic material, such as dewatered sewage sludge, mixed organic waste, and animal waste. The method includes mixing the infectious waste organic material with an organic fibrous material that is comminuted in order to provide a reaction mixture. An oxidizing agent is an optional additive. The reaction mixture is heated in a hyperbaric reactor vessel at elevated pressure and temperature for a period of time sufficient to create saturated steam and produce a substantially denatured product containing inactive pathogens. The denatured product is dehydrated to produce a solid, free-flowing product that can be used in a variety of agricultural, industrial, or commercial applications. The odor is controlled so that malodorous compounds are not released into the atmosphere. That invention differs completely in application and formulation from the invention disclosed herein.

Biohazardous wastes contain microorganisms that can be harmful to the general population and the environment, so methods exist which enable these pathogens to be eliminated. However, these methods are not completely inert to the environment, since they are associated with indirect risks to the health of the population resulting from the release of different toxic contaminants. Likewise, large products from this type of waste continue to generate urban waste that cannot be integrated or reused in a circular structure. Alternatives to incineration such as an ecologically innocuous chemical treatment would enable this type of waste to be reused as raw material for the generation of composts and/or other products that require organic sources. These two characteristics have not appeared simultaneously in any chemical treatment, since some offer efficiency in the elimination of microorganisms; however, they compromise the decomposition of the waste, which makes the reuse of this waste impossible, thus preventing a circular process from being adopted and generating an ecological impact due to the production of industrial waste.

On the other hand, “in ovo” vaccination is a relatively recent technique, having been described for the first time in 1982, and with the first machine marketed by an Embrex company appearing in 1992, although other machines were developed later by different companies. This technique is used for the injection of certain live vaccines immediately before the eggs are transferred from the incubation trays to the hatchers at 17-19 days.

The method is widely used throughout the world at present, especially for broilers and, to some extent, in breeders for vaccination against Marek’s and Gumboro’s diseases. It is suitable for the administration of multivalent and recombinant vaccines against MDV, IBDV, FPV, NDV, ILT, etc.; these aspects are being researched and could lead to the large-scale application of other antigens. Normally a small hole is made in the blunt end of the eggshell, and the vaccine is injected under the chorioallantoic membrane.

Currently, hundreds of vaccines for veterinary and human use are produced in ovo. This process involves the use of fertilized chicken eggs, which are injected with active and inactive viruses so that they proliferate as the chicken embryo develops. The fluid containing the viruses is harvested from the eggs. For some injectable vaccines, such as those against influenza, the viruses are inactivated and the virus antigen is purified. The production process continues with the purification and testing stages. This entire process generates large amounts of biohazardous waste as classified according to the Official Mexican Standard NOM-087-SEMARNAT-SSA1-2002, such as cultures and strains of biohazardous and pathological agents and non-anatomical wastes.

It is the object of the invention to provide a formulation which, in association with a grinding system, transforms biohazardous waste in the production of vaccines in ovo to raw material for the preparation of composts, making possible:
the inactivation of biohazardous waste for reuse,
the inactivation of industrial biohazardous waste for reuse as a protein source for the preparation of protein products,
a formulation that can be applied for the purpose of eliminating microorganisms in wastes from the production of drugs and vaccines in ovo with applications in:
   biohazardous wastes,
   waste of animal origin with a high protein content and potential for putrefaction for the preparation of composts.
The technical details of the developed embodiment are described below.

### BRIEF DESCRIPTION OF THE FIGURES

FIG. 1 shows the waste destruction process with the parameters used: 65 kg of waste from the production of vaccines in ovo in yellow bags, 700 ml of peracetic acid, in 10 liters of water for a 20-minute cycle. As a result, 50 liters of leachate is obtained which passes through the drain, as well as 15 kg of pathogen-free solid waste that is ready to be reused.

FIG. 2 shows the monitoring of cell cultures exposed to supernatants treated with peracetic acid/hydrogen peroxide, negative control (not exposed to supernatant) and positive control (exposed to untreated supernatant). The columns indicate the culture time, and the rows indicate the type of exposure.

FIG. 3 shows the monitoring of MCF7 (Michigan Cancer Foundation 7; this is a breast cancer cell line isolated in 1970 from a 69-year-old Caucasian woman), negative control (A-D), positive control exposed to the supernatant obtained from the biohazardous waste bags prior to processing (E-H), and post-process leaching (I-H). Arrows indicate cytopathic damage (cell death).

FIG. 4 shows the monitoring of HEPG2 (hepatocellular carcinoma), negative control (A-D), positive control exposed to the supernatant obtained from the biohazardous waste bags prior to processing (E-H), post-process leaching (I-H), and the cells exposed to the solids obtained after the integral grinding and crushing treatment that simultaneously disinfects the biohazardous waste, reducing its volume by up to 90% and rendering it harmless for disposal as ordinary waste. Arrows indicate cytopathic damage (cell death).

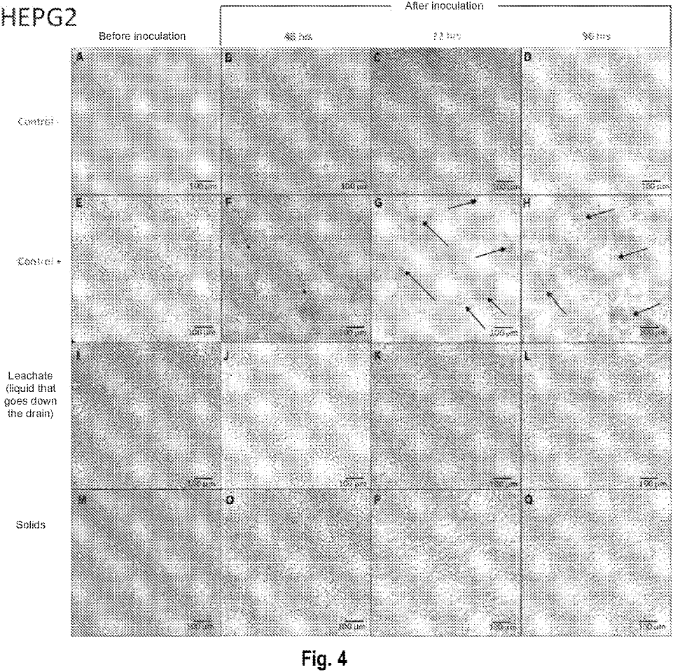

FIG. 5 shows the histogram of cell viability based on the result of the MTT assay, which is a method for determining the possible cytotoxic effect of an agent on tumor cell lines or primary cultures of normal cells; the Y axis represents the percentage of viability of each well, and the X axis represents the type of treatment and control. The bars have their standard deviation.

### DETAILED DESCRIPTION OF THE INVENTION

The characteristic details of the peracetic acid-based formulation associated with a grinding process, the combination of which transforms the crops and strains of biohazardous waste generated in the production of vaccines “in ovo” into raw material for the preparation of high-protein composts are unambiguously demonstrated in the following description and in the accompanying illustrative drawings.

For the application of the formulation, a grinding process was developed for the treatment of waste which includes the combination of the process and the peracetic acid-based formulation that transforms biohazardous waste into special waste. This process consists of grinding and chemical disinfection (reaction mixture) under controlled conditions for time and the concentration of the chemical sterilization solution, whereupon the volume of the waste is reduced by destroying the waste until it is unrecognizable and pathogens are rendered inactive. As a result of the process, leachate is obtained having characteristics that enable it to be disposed of in general drainage, as well as transformed solid waste that is free of pathogens and can be treated as special waste.

The waste destruction process consists of the following stages: loading, grinding, chemical washing, drainage, and solids output. In the loading stage, the biohazardous waste generated in the production of vaccines in ovo is introduced, and sprinklers carry out a first rinse in the upper chamber of the equipment. During grinding, the waste is destroyed and then exposed to a chemical wash through the action of a mixture of water with peracetic acid. Finally, the leachate comes out through a drain valve, and the sterilized solids are removed by means of a screw conveyor. This solid waste is collected in containers for subsequent reuse.

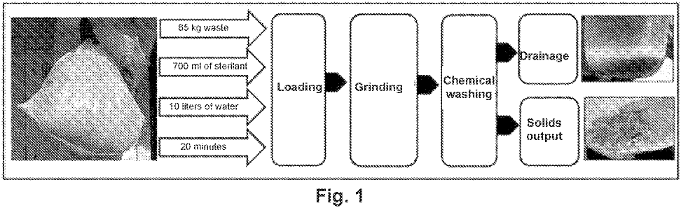

FIG. 1 shows the waste destruction process with the parameters used: 65 kg of waste from the production of vaccines in ovo in yellow bags, 700 ml of peracetic acid, in 10 liters of water for a 20-minute cycle. After the aforementioned stages, 50 liters of leachate are obtained, which pass through the drain, as well as 15 kg of pathogen-free solid waste that is ready to be reused.

In order to reuse biohazardous waste as raw material for composts and/or products that require an organic source, the complete inactivation of the waste must be verified, meaning that it must not have any pathogenic microorganism that continues to be considered a biohazardous waste. Therefore, between 72,000 to 88,000 parts per million (PPM) or 72 to 88 grams per liter of peracetic acid are obtained upon producing the mixture of hydrogen peroxide, acetic acid, and sulfuric acid. These components are found in the proportions of 40-80% (hydrogen peroxide), 10-40% (acetic acid), and 1-10% (sulfuric acid), with acetic acid (CH₃COOH) and hydrogen peroxide (H₂O₂) reacting to form peracetic acid, and with the presence of sulfuric acid (H₂SO₄) stabilizing this molecule. Its stable formula is as follows:

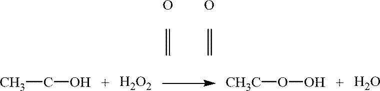

This solution was diluted to 1 and 0.5% volume/volume with potable water. This yielded solutions of 200 to 400 ppm or 200 to 400 mg per liter of peracetic acid. These solutions were used to validate their virucidal capacity in industrial biohazardous wastes contaminated with New Castle-type viruses from manufacturing vaccines in ovo.

### Cell Culture Monitoring

A cell culture of HEK 293 cells (human embryonic kidney 293 cells) distributed in a 24-well plate was monitored for 96 hours. During the first 24 hours after the exposure of the supernatant liquid to the biohazardous waste, untreated and treated with the sterilizing solution, no cytopathic evidence was found, as can be seen in FIGS. 1A, 1G, 1K, 1O, 1S. The culture in the different wells of the controls and test samples continued to grow and proliferate as normal; however, at 72 hours the positive control wells that were exposed to the supernatant liquid of the waste without treatment with the sterilizing liquid began to manifest signs of cytopathy, as is shown in FIG. 1D. Finally, at 96 hours, severe cytopathic effects were observed, which are shown in FIG. 1E, whereas the other cultures exhibited normal growth, similar to that of the negative control (non-exposed cells).

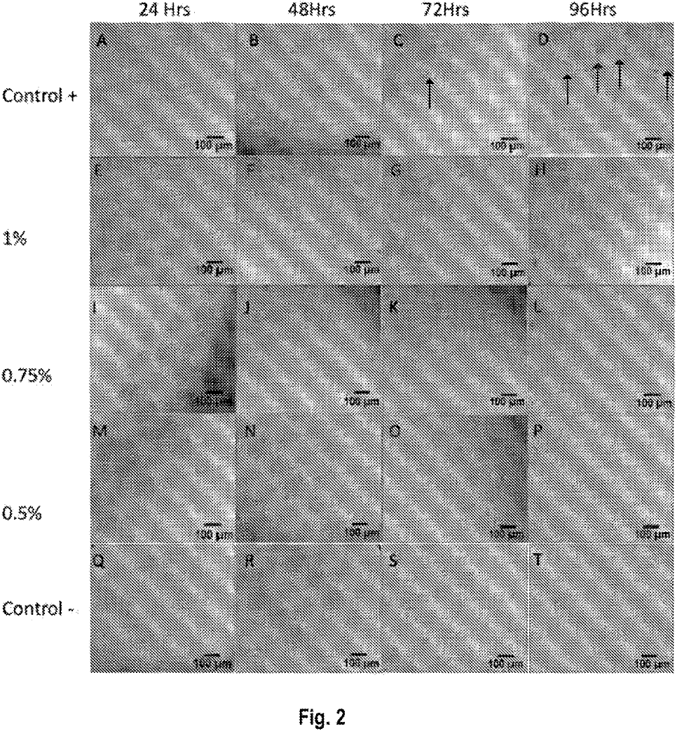

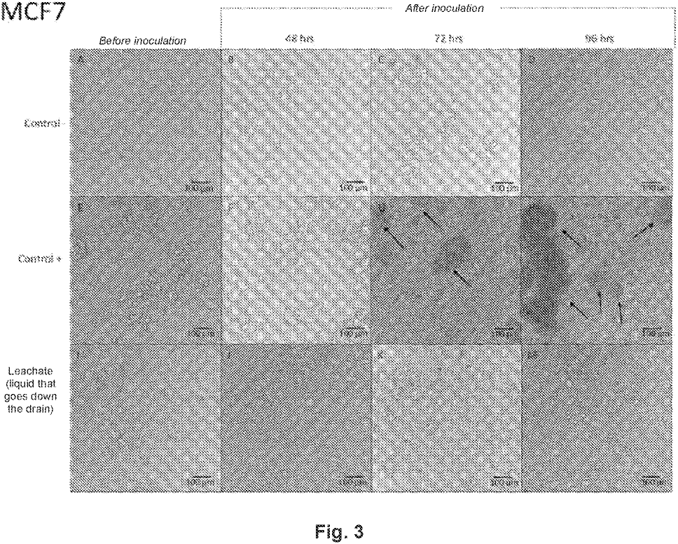

On the other hand, HEPG2 and MCF7 cells exposed to leachate and solids only for HEPG2 exhibit a behavior similar to that of HEK293, with the exposed cultures not showing any signs of cytopathy, just like the negative control; this is shown in FIGS. 2 and 3. However, the cytopathic evidence is clear in the positive controls from 72 and 48 hours, respectively, which can be seen in FIG. 2F and FIG. 3C.

### MTT Assay

After MTT processing, FIG. 2 shows that the cells that were exposed to the viral supernatant have a viability of 80-95% compared to the negative control. They do not mimic the behavior of the negative control, since the neutralization process and the possible salts that are formed might affect the cell culture, inducing cell death due to an osmotic imbalance.

However, the positive control has a viability of 45%, showing cell death due to the presence of the virus and the replication and proliferation thereof. In order to avoid having a low cell viability in the negative control that might affect the reading, the culture was not continued for another 24 hours. For HEPG2 and MCF7, MTT was not necessary, since cell death was evident at 96 hours, which can be seen in FIGS. 2D, 2M and FIGS. 3D, 3L, 3G.

### Process Validation and Formulation With Waste With Other Microorganisms

The bactericidal and fungicidal action was verified using five different species: Escherichia coli, Pseudomona aureginosa representing the bacteria in the Gram-negative group, Staphylococcus aureus, Bacillus subtilius representing the bacteria in the Gram-positive group, and Candida afficans as a fungus. These microorganisms were allowed to grow on nutrient media to proliferation. After 24 hours, 3 batches of waste prepared with sterile residue from needles, syringes, sheets, compresses, and cotton pads were contaminated. Samples were taken from the various proliferating suspensions, with dilutions up to 10×⁻⁶ being prepared in triplicate. Each batch was then eliminated in the system with concentrations of 0.5 and 1% volume/volume for 5 minutes, respectively (washing). At the end of each process, 3 ml of the liquid sample were neutralized with sodium hydroxide. Subsequently, each sample was taken for subsequent culturing in specific agar solutions for each microorganism; these were allowed to incubate for 24 hours. After incubation, the colony-forming units (CFUs) were counted for purposes of quantification.

A clear elimination of pathogens from 6Log₁₀ to 7Log₁₀ was observed among all species, leaving the cultures exposed to the practical acid solution with no evidence of any colony formation. Formed colonies were observed in the positive controls, indicating the proliferation of the microorganism.

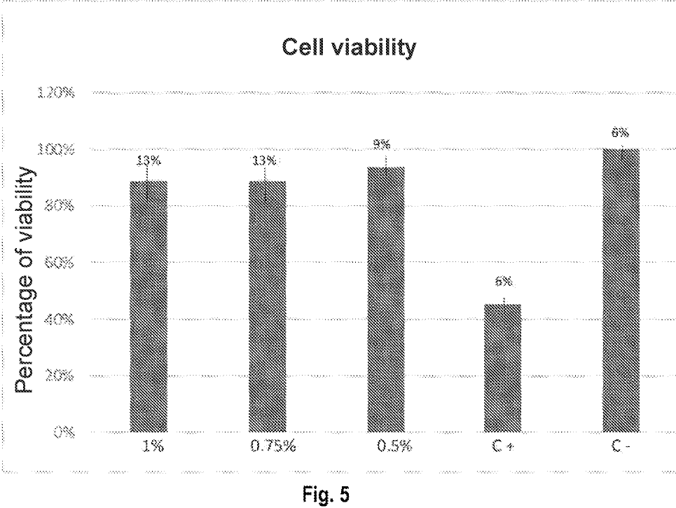

FIG. 5 shows the cell viability based on the result of the MTT assay.

The sterilizing liquid and the grinding process show efficacy in the elimination of the New Castle virus from the biohazardous waste that was discarded in the in ovo vaccine production process, and the absence of other microorganisms that might cause these wastes to be categorized as biohazards was likewise confirmed. The cultures that were inoculated with the treated and neutralized supernatant behaved like a healthy culture, whereas the control that was inoculated with the untreated supernatant fluid exhibited signs of mild (at 72 hours) to severe (94 hours) cytopathy. Likewise, the presence of other microorganisms such as bacteria and fungi was verified, demonstrating that the combination of peracetic acid and a grinding system is a binomial for the transformation of biohazardous waste to special waste; transformation of biohazardous waste to special waste by means of chemical washing to inactivate the viral proliferation of biohazardous wastes from the production of vaccines in ovo and enable this product to be labeled as raw material for the preparation of composts and/or products that require organic sources.

### Resulting Formulation

The formulation that was obtained as a result of all the analyses described and yields the best results is:

A formulation for the transformation of biohazardous waste to raw material for the preparation of composts, comprising:
a. peracetic acid for the elimination of microorganisms in biohazardous wastes from the production of drugs and vaccines in ovo, comprising:
   i. peracetic acid at 23% weight/weight in water, which is obtained by mixing hydrogen peroxide, acetic acid, and sulfuric acid;
   ii. the proportions of the above components are:
      1. hydrogen peroxide 40-80%,
      2. acetic acid 10-40%, and
      3. sulfuric acid 1-10%;
   iii. wherein acetic acid (CH₃COOH) and hydrogen peroxide (H₂O₂) react to form peracetic acid, with the presence of sulfuric acid (H₂SO₄) stabilizing this molecule;
   iv. the solution is diluted in 1% hydrogen peroxide, 0.75% acetic acid, and 0.5% sulfuric acid volume/volume with potable water;
   v. all of the above yields solutions of 200 to 400 ppm or the equivalent thereof of 200 to 400 mg per liter of peracetic acid.

In addition to the above, it is combined with a grinding process for biohazardous waste that produces a reaction mixture in order to increase the biocidal action of the PAA formulation.

The preceding description of the disclosed definitions is provided in order to enable any person skilled in the art to implement or use the present invention. Various modifications to the generic definitions and/or implementations defined herein may be applied to other embodiments without departing from the spirit or scope of the invention. Therefore, the present invention is not intended to be limited to the embodiments shown herein, but should be granted the broadest scope consistent with the following claims and the principles and novel features disclosed herein.

1-3. (canceled)

4. A peracetic acid-based solution comprising:
hydrogen peroxide in an amount from about 40-80%;
acetic acid in an amount from about 10-40%; and
sulfuric acid in an amount from about 1-10%,
wherein the acetic acid (CH₃COOH) and the hydrogen peroxide (H₂O₂) react to form peracetic acid, with the presence of the sulfuric acid (H2SO4) stabilizing a molecule.

5. The solution of claim 4, wherein the solution is diluted in 1% hydrogen peroxide, 0.75% acetic acid, and 0.5% sulfuric acid volume/volume with potable water.

6. The solution of claim 4, wherein the solution is at a concentration of about 200 to 400 ppm or the equivalent thereof of peracetic acid.

7. The solution of claim 4, wherein the solution enables at least one of a bactericidal, a fungicidal, and a viricidal action.

8. The solution of claim 4, wherein the solution enables destruction of biohazardous waste for reuse as raw material in the preparation of a protein product.

9. The solution of claim 8, wherein the biohazardous waste results from the production of vaccines “in ovo.”.

10. The solution of claim 8, wherein the raw material is a protein source.

11. The solution of claim 8, wherein the protein product is a high-protein compost.

12. The solution of claim 8, wherein the solution reduces the volume of the biohazardous waste and inactivates pathogens in the biohazardous waste.

13. The solution of claim 4, wherein the solution enables destruction of waste of animal origin with a high protein content.

14. A method of biohazardous waste destruction using waste destruction equipment, the method comprising:
introducing the biohazardous waste into the waste destruction equipment;
performing a first rinse on the biohazardous waste;
destroying the biohazardous waste, wherein destroying the biohazardous waste includes:
grinding and crushing the biohazardous waste by the waste destruction equipment;
exposing the biohazardous waste to a chemical wash, wherein the chemical wash incudes a peracetic acid-based solution; and
transforming the biohazardous waste into special waste, wherein the special waste includes leachate and solid waste; and
collecting the special waste from the destroyed biohazardous waste, wherein the leachate is released through a drain valve of the waste destruction equipment and the solid waste is collected in a container.

15. The method of claim 14, wherein the biohazardous waste results from the production of vaccines “in ovo.”.

16. The method of claim 14, wherein performing the first rinse on the biohazardous waste includes rinsing the biohazardous waste in an upper chamber of the waste destruction equipment.

17. The method of claim 14, further comprising removing the solid waste from the waste destruction equipment with a screw conveyor.

18. The method of claim 14, wherein exposing the biohazardous waste to a chemical wash includes at least one of a bactericidal, a fungicidal, and a viricidal action.

19. The method of claim 14, wherein destroying the biohazardous waste includes reducing the volume of the biohazardous waste and inactivating pathogens in the biohazardous waste.

20. The method of claim 14, further comprising preparing the special waste for reuse as a raw material in the preparation of a protein product.

21. The method of claim 20, wherein the raw material is a protein source, and wherein the protein product is a high-protein compost.

22. The method of claim 14, further comprising destroying waste of animal origin with a high protein content.

23. A method of waste destruction using waste destruction equipment, the method comprising:
introducing the waste to the waste destruction equipment;
destroying the waste, wherein destroying the waste includes:
grinding and crushing the waste by the waste destruction equipment;
exposing the waste to a chemical wash, wherein the chemical wash incudes a peracetic acid-based solution; and
transforming the waste into special waste, wherein the special waste includes leachate and solid waste; and
collecting the special waste from the destroyed biohazardous waste.
